(19) United States
(12) Patent Application Publication
(10) Pub. No.: US 2010/0099663 A1
(43) Pub. Date: Apr. 22, 2010

(54) 4-TRIFLUOROMETHOXYPHENOXYBENZOL-4'-SULFONIC ACIDS, METHOD FOR THE PRODUCTION AND USE THEREOF IN MEDICAMENTS

(75) Inventors: Manfred SCHUDOK, Eppstein (DE); Armin HOFMEISTER, Dexheim (DE)

Correspondence Address: ANDREA Q. RYAN;SANOFI-AVENTIS U.S. LLC, 1041 ROUTE 202-206, MAIL CODE: D303A, BRIDGEWATER, NJ, 08807, US

(73) Assignee: SANOFI-AVENTIS DEUTSCHLAND GMBH, Frankfurt am Main (DE)

(21) Appl. No.: 12/643,162

(22) Filed: Dec. 21, 2009

Related U.S. Application Data
(63) Continuation of application No. 11/611,199, filed on Dec. 15, 2006, now abandoned.
Application No. 11/611,199 is a continuation of application No. PCT/EP2005/006416, filed on Jun. 15, 2005.

(30) Foreign Application Priority Data
Jun. 30, 2004 (DE) 102004031620.1

(51) Int. Cl. A61K 31/55; C07D 217/22; A61K 31/47; C07D 471/02; A61K 31/4353; C07D 223/16; A61P 43/00

(52) U.S. Cl. 514/213.01; 546/141; 514/309; 546/115; 514/302; 540/593

(57) ABSTRACT

The present invention is directed to a compound of formula I,

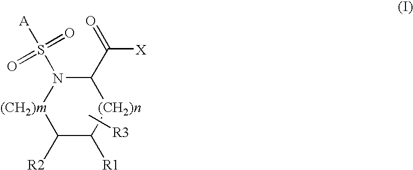

wherein R1, R2, R3, X, A, m and n are as herein defined, the process for its preparation and its use thereof as a medicament.

This application is a Continuation of International Application No. PCT/EP2005/006416, filed Jun. 15, 2005, and of U.S. patent application Ser. No. 11/611,199, filed Dec. 15, 2006.

### FIELD OF THE INVENTION

The invention relates to novel derivatives of 4-trifluoromethoxyphenoxybenzene such as 4-trifluoromethoxyphenoxybenzene-4′-sulfonic acid, the respective sulfonyl chloride, derivatives such as sulfonamides, and processes for their preparation and use thereof as medicaments.

### BACKGROUND OF THE INVENTION

Pharmacologically active substances are frequently composed of one or more ring systems. These may be saturated or unsaturated carbocycles or heterocycles. A particular spatial arrangement is necessary for exercising the biological activity. In addition, there is a whole series of further different but very important interactions which contribute to a binding affinity. Possible examples are pi-pi interactions of aromatic systems between protein and inhibitor, ionic interactions, or acid-base interactions. Functional groups are responsible in particular for the latter. These are often “attached” to the abovementioned ring systems. However, the biological activity is only one aspect which must be satisfied by active substances which are to be developed as potential medicaments. Another important area, which has often been underestimated in the past, is to be seen in the absorption, distribution, metabolism and excretion of the active substance. Often single parts of the molecule are particularly responsible for differences in behavior of the molecules in this area, in just the same way as for the biological activity. Once again, ring systems, their particular properties and functional groups may be involved. However, it is in many cases not possible satisfactorily to correlate with, for example, particular physicochemical properties of these groups, or only a suggestive prediction is to date possible by methods of computational chemistry, in contrast to the area of the biological activity. Particular complexity emerges when small modifications are made to these functional groups and lead to very strong effects. By this is meant that, for example, there may be significant changes in absorption, distribution (equal to disposition), metabolism and excretion. Thus, it is perfectly possible for a lead structure with inadequate properties to become a candidate for development.

It has now been found, surprisingly, that compounds which comprise radicals of the invention have distinctly better pharmacokinetic properties than very closely related compounds which have simple alkyl ether or alkyl fluoride side chains. Improved pharmacokinetic properties mean in this connection that there are observed to be both higher maximally achievable plasma levels and longer half-lives. This means that a beneficial influence thus takes place in particular on absorption, metabolism and excretion. At the same time, for example, the sulfonamides of the invention which are often to be found in active substances and which are prepared from the previously unknown sulfonyl chlorides of the invention are novel. The same applies to the corresponding sulfonic acids.

The compounds of the invention can be employed widely. For example, matrix metalloproteinase inhibitors (MMP) frequently comprise side chains similar to the type of the invention. Cyclic and, in particular, bicyclic basic structures are widely described. For example, WO 97/18194 describes tetrahydroisoquinoline derivatives, and WO 03/016248 describes further heterocycles.

### SUMMARY OF THE INVENTION

The invention therefore relates to a compound of the formula I

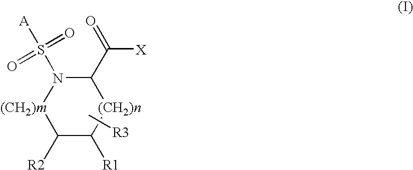

and/or all stereoisomeric forms of the compound of the formula I and/or mixtures of these forms in any ratio, and/or a physiologically tolerated salt of the compound of the formula I, where

X is —OH or NH—OH,

A is a radical of the formula II

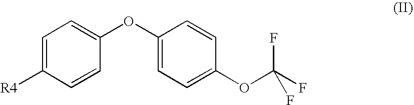

in which R4 means the covalent bond to the S atom of the formula I, R1, R2 and R3 are identical or different and are independently of one another
1) hydrogen atom,
2) —(C₁-C₆)-alkyl in which alkyl is unsubstituted or substituted once or twice by —(C₃-C₆)-cycloalkyl, —(C₂-C₄)-alkenylene, —(C₂-C₆)-alkynyl, —(C₆-C₁₄)-aryl or Het ring,
3) —C(O)—O—R8 in which R8 is
   3)1) hydrogen atom,
   3)2) —(C₁-C₆)-alkyl in which alkyl is unsubstituted or substituted once or twice by —(C₃-C₆)-cycloalkyl, —(C₂-C₄)-alkenylene, —(C₂-C₆)-alkynyl, —(C₆-C₁₄)-aryl, or Het ring or once to five times by fluorine,
   3)3) —(C₆-C₁₄)-aryl or
   3)4) Het ring,
4) —O—R8 in which R8 has the abovementioned meaning,
5) —(C₃-C₆)-cycloalkyl,
6) -halogen,
7) —NO₂, or
8) —CN, or
9) R1 and R2 form together with the carbon atoms to which they are bonded a —(C₆-C₁₄)-aryl ring in which the ring is unsubstituted or substituted once or twice by G,
10) R1 and R2 form together with the carbon atoms to which they are bonded a —(C₅-C₇)-cycloalkyl ring in which the ring is unsubstituted or substituted once or twice by G, or
11) R1 and R2 form together with the carbon atoms to which they are bonded a 5-, 6- or 7-membered Het ring, where the ring is unsubstituted or substituted once by G, or
12) R1 and R2 form together with the carbon atoms to which they are bonded an indolyl in which the indolyl is unsubstituted or substituted once or twice by G,

G is
1) hydrogen atom,
2) halogen,
3) ═O,
4) —(C₁-C₆)-alkyl in which alkyl is unsubstituted or substituted once, twice or three times by halogen, —(C₃-C₆)-cycloalkyl, —(C₂-C₄)-alkenylene, —(C₂-C₆)-alkynyl, —(C₆-C₁₄)-aryl or Het ring,
5) —(C₆-C₁₄)-aryl,
6) Het ring,
7) —C(O)—O—R10 in which R10 is
   a) —(C₁-C₆)-alkyl in which alkyl is unsubstituted or substituted once or twice by —(C₃-C₆)-cycloalkyl, —(C₂-C₄)-alkenylene, —(C₂-C₆)-alkynyl, —(C₆-C₁₄)-aryl or Het ring,
   b) —(C₆-C₁₄)-aryl or
   c) Het ring
8) —C(S)—O—R10 in which R10 is as defined above,
9) —C(O)—NH—R11 in which R11 is
   a) —(C₁-C₆)-alkyl in which alkyl is unsubstituted or substituted once or twice by —(C₃-C₆)-cycloalkyl, —(C₆-C₁₄)-aryl or Het ring, or
   b) —(C₆-C₁₄)-aryl or
   c) Het ring,
10) —C(S)—NH—R11 in which R11 is as defined above,
11) —O—R12 in which R12 is
   a) hydrogen atom,
   b) —(C₁-C₆)-alkyl in which alkyl is unsubstituted or substituted once, twice or three times by halogen, —(C₃-C₆)-cycloalkyl, —(C₂-C₄)-alkenylene, —(C₂-C₆)-alkynyl, —(C₆-C₁₄)-aryl or Het ring,
   c) —(C₆-C₁₄)-aryl,
   d) Het ring,
   e) —C(O)—O—R13 in which R13 is
      e)1) —(C₁-C₆)-alkyl in which alkyl is unsubstituted or substituted once or twice by —(C₃-C₆)-cycloalkyl, —(C₂-C₄)-alkenylene, —(C₂-C₆)-alkynyl, —(C₆-C₁₄)-aryl, or Het ring, or
      e)2) —(C₆-C₁₄)-aryl or
      e)3) Het ring,
   f) —C(S)—O—R13 in which R13 is as defined above,
   g) —C(O)—NH—R14 in which R14 is
      g)1) —(C₁-C₆)-alkyl in which alkyl is unsubstituted or substituted once or twice by —(C₃-C₆)-cycloalkyl, —(C₂-C₄)-alkenylene, —(C₂-C₆)-alkynyl, —(C₆-C₁₄)-aryl or Het ring, or
      g)2) —(C₆-C₁₄)-aryl or
      g)3) Het ring, or
   h) —C(S)—NH—R14 in which R14 is as defined above,
12) —C(O)—R10 in which R10 is as defined above,
13) —S(O)ₚ—R12 in which R12 is as defined above, and p is the integers zero, 1 or 2,
14) —NO₂,
15) —CN or
16) —N(R15)-R12 in which R15 is
   16)1) hydrogen atom,
   16)2) —(C₁-C₆)-alkyl or
   16)3) SO₂—(C₁-C₆)-alkyl in which alkyl is unsubstituted or substituted once or twice by —(C₃-C₆)-cycloalkyl, —(C₂-C₄)-alkenylene, —(C₂-C₆)-alkynyl, —(C₆-C₁₄)-aryl or Het ring, and R12 is as defined above, or
17) —SO₂—N(R12)-R16 in which R12 is as defined above, and in which R16 is
   17)1) hydrogen atom,
   17)2) —(C₁-C₆)-alkyl in which alkyl is unsubstituted or substituted once or twice by —(C₃-C₆)-cycloalkyl, —(C₂-C₄)-alkenylene, —(C₂-C₆)-alkynyl, —(C₆-C₁₄)-aryl or Het ring,
   17)3) —C(O)—O—R8 in which R8 has the above-mentioned meaning,
   17)4) —O—R8 in which R8 has the abovementioned meaning, or
   17)5) —(C₃-C₆)-cycloalkyl, and
      m and n are identical or different and are the numbers zero, 1, 2 or 3, with the proviso that the total of m and n amounts to zero, 1, 2 or 3.

### DETAILED DESCRIPTION OF THE INVENTION

The invention further relates to the compounds of the formula I, where

X is —OH or —NH—OH,

A is a radical of the formula II,

R1 and R2 form together with the carbon atoms to which they are bonded
a) a —(C₆-C₁₄)-aryl ring in which aryl is a radical from the series phenyl, naphthyl, 1-naphthyl, 2-naphthyl, anthryl or fluorenyl, and is unsubstituted or substituted once or twice by G, or
b) a 5-, 6- or 7-membered Het ring in which Het ring is a radical from the series furan, imidazole, isoxazole, oxazole, pyrazine, pyrazole, pyridazine, pyridine, pyrimidine, thiazole or thiophene, and is unsubstituted or substituted once or twice by G, or
c) an indolyl in which the indolyl is unsubstituted or substituted once or twice by G,

G is
1) hydrogen atom,
2) fluorine, chlorine, bromine or iodine,
3) ═O,
4) —(C₁-C₆)-alkyl in which alkyl is unsubstituted or substituted once, twice or three times by halogen, —(C₃-C₆)-cycloalkyl, —(C₂-C₆)-alkynyl, —(C₆-C₁₄)-aryl, in which aryl is phenyl or naphthyl, or Het ring in which the Het ring is a radical from the series acridinyl, azepinyl, azetidinyl, aziridinyl, benzimidazalinyl, benzimidazolyl, benzofuranyl, benzothiofuranyl, benzothiophenyl, benzoxazolyl, benzthiazolyl, benztriazolyl, benztetrazolyl, benzisoxazolyl, benzisothiazolyl, carbazolyl, 4aH-carbazolyl, carbolinyl, quinazolinyl, quinolinyl, 4H-quinolizinyl, quinoxalinyl, quinuclidinyl, chromanyl, chromenyl, cinnolinyl, decahydroquinolinyl, dibenzofuranyl, dibenzothiophenyl, dihydrofuran[2,3-b]-tetrahydrofuranyl, dihydrofuranyl, dioxolyl, dioxanyl, 2H, 6H-1,5,2-dithiazinyl, furanyl, furazanyl, imidazolidinyl, imidazolinyl, imidazolyl, 1H-indazolyl, indolinyl, indolizinyl, indolyl, 3H-indolyl, isobenzofuranyl, isochromanyl, isoindazolyl, isoindolinyl, isoindolyl, isoquinolinyl (benzimidazolyl), isothiazolidinyl, 2-isothiazolinyl, isothiazolyl, isoxazolyl, isoxazolidinyl, 2-isoxazolinyl, morpholinyl, naphthyridinyl, octahydroisoquinolinyl, oxadiazolyl, 1,2,3-oxadiazolyl, 1,2,4-oxadiazolyl, 1,2,5-oxadiazolyl, 1,3,4-oxadiazolyl, oxazolidinyl, oxazolyl, oxazolidinyl, oxothiolanyl, pyrimidinyl, phenanthridinyl, phenanthrolinyl, phenazinyl, phenothiazinyl, phenoxathiinyl, phenoxazinyl, phthalazinyl, piperazinyl, piperidinyl, pteridinyl, purynyl, pyranyl, pyrazinyl, pyroazolidinyl, pyrazolinyl, pyrazolyl, pyridazinyl, pyridooxazolyl, pyridoimidazolyl, pyridothiazolyl, pyridothiophenyl, pyridinyl, pyridyl, pyrimidinyl, pyrrolidinyl, pyrrolinyl, 2H-pyrrolyl, pyrrolyl, tetrahydrofuranyl, tetrahydroisoquinolinyl, tetrahydroquinolinyl, tetrahydropyridinyl, 6H-1,2,5-thiadazinyl, 1,2,3-thiadiazolyl, 1,2,4-thiadiazolyl, 1,2,5-thiadiazolyl, 1,3,4-thiadiazolyl, thianthrenyl, thiazolyl, thienyl, thienothiazolyl, thienooxazolyl, thienoimidazolyl, thiomorpholinyl, thiophenyl, triazinyl, 1,2,3-triazolyl, 1,2,3-triazolyl, 1,2,4-triazolyl, 1,2,5-triazolyl, 1,3,4-triazolyl and xanthenyl,
5) —(C₆-C₁₄)-aryl in which aryl is phenyl or naphthyl,
6) Het ring in which Het is as defined above,
7) —C(O)—O—R10 in which R10 is
   a) —(C₁-C₆)-alkyl in which alkyl is unsubstituted or substituted once or twice by —(C₃-C₆)-cycloalkyl, —(C₂-C₆)-alkynyl, —(C₆-C₁₄)-aryl, or Het ring, in which aryl is phenyl or naphthyl, and Het is as defined above,
   c) —(C₆-C₁₄)-aryl in which aryl is phenyl or naphthyl, or
   d) Het ring in which Het is as defined above,
8) —C(S)—O—R10 in which R10 is as defined above,
9) —C(O)—NH—R11 in which R11 is
   a) —(C₁-C₆)-alkyl in which alkyl is unsubstituted or substituted once or twice by —(C₃-C₆)-cycloalkyl, —(C₆-C₁₄)-aryl or Het ring, in which aryl is phenyl or naphthyl, and Het is as defined above, or
   b) —(C₆-C₁₄)-aryl in which aryl is phenyl or naphthyl, or
   c) Het ring in which Het is as defined above,
10) —C(S)—NH—R11 in which R11 is as defined above,
11) —O—R12 in which R12 is
   a) hydrogen atom,
   b) —(C₁-C₆)-alkyl in which alkyl is unsubstituted or substituted once, twice or three times by halogen, —(C₃-C₆)-cycloalkyl, —(C₂-C₆)-alkynyl, —(C₆-C₁₄)-aryl or Het ring, in which aryl is phenyl or naphthyl, and Het is as defined above,
   c) —(C₆-C₁₄)-aryl in which aryl is phenyl or naphthyl,
   d) Het ring in which Het is as defined above,
   e) —C(O)—O—R13 in which R13 is
      e)1) —(C₁-C₈)-alkyl in which alkyl is unsubstituted or substituted once or twice by —(C₃-C₆)-cycloalkyl, —(C₂-C₆)-alkynyl, —(C₆-C₁₄)-aryl or Het ring, in which aryl is phenyl or naphthyl, and Het is as defined above,
      e)2) —(C₆-C₁₄)-aryl in which aryl is phenyl or naphthyl, or
      e)3) Het ring in which Het is as defined above,
   f) —C(S)—O—R13 in which R13 is as defined above,
   g) —C(O)—NH—R14 in which R14 is
      g)1) —(C₁-C₆)-alkyl in which alkyl is unsubstituted or substituted once or twice by —(C₃-C₆)-cycloalkyl, —(C₂-C₆)-alkynyl, —(C₆-C₁₄)-aryl or Het ring, in which aryl is phenyl or naphthyl, and Het is as defined above,
      g)2) —(C₆-C₁₄)-aryl in which aryl is phenyl or naphthyl, or
      g)3) Het ring in which Het is as defined above, or
   h) —C(S)—NH—R14 in which R14 is as defined above,
12) —C(O)—R10 in which R10 is as defined above,
13) —S(O)ₚ—R12 in which R12 is as defined above, and p is the integers zero, 1 or 2,
14) —NO₂,
15) —CN,
16) —N(R15)-R12 in which R15 is
   16)1) hydrogen atom,
   16)2) —(C₁-C₆)-alkyl or
   16)3) —SO₂—(C₁-C₆)-alkyl in which alkyl is unsubstituted or substituted once or twice by —(C₃-C₆)-cycloalkyl, —(C₂-C₆)-alkynyl, —(C₆-C₁₄)-aryl or Het ring, in which aryl is phenyl or naphthyl, and Het is as defined above, and R12 is as defined above, or
17) —SO₂—N(R12)-R16 in which R12 is as defined above, and in which R16 is
   17)1) hydrogen atom,
   17)2) —(C₁-C₆)-alkyl in which alkyl is unsubstituted or substituted once or twice by —(C₃-C₆)-cycloalkyl, —(C₂-C₆)-alkynyl, —(C₆-C₁₄)-aryl or Het ring, in which aryl is phenyl or naphthyl, and Het is as defined above,
   17)3) —C(O)—O—R8 in which R8 has the above-mentioned meaning,
   17)4) —O—R8 in which R8 has the abovementioned meaning, or
   17)5) —(C₃-C₆)-cycloalkyl, and

m is the number 1 or 2, and n is the number zero.

m is the number 1, and n is the number two, or

m and n are identical and each is the number 1.

The invention further relates to the compound of the formula I where
X is —OH or —NH—OH,
A is a radical of the formula II,
m is the number 1 and n is the number two, or
m and n are identical and each is the number 1, and
R1 and R2 form together with the carbon atoms to which they are bonded a phenyl, tetrahydrofuranyl or cyclohexyl.

The invention further relates to the compound of the formula I from the series
2-[4-(4-trifluoromethoxyphenoxy)benzenesulfonyl]-1,2,3,4-tetrahydroisoquinoline-1-hydroxycarboxamide,
2-[4-(4-trifluoromethoxyphenoxy)benzenesulfonyl]decahydroisoquinoline-1-(N-hydroxy)carboxamide,
5-[4-(4-trifluoromethoxyphenoxy)benzenesulfonyl]octahydrofuro[3,2-c]pyridine-4-carboxylic acid,
5-[4-(4-trifluoromethoxyphenoxy)benzenesulfonyl]octahydrofuro[3,2-c]pyridine-4-(N-hydroxy)carboxamide or
2-[4-(4-trifluoromethoxyphenoxy)benzenesulfonyl]decahydrobenzo[c]azepine-1-(N-hydroxy)carboxamide.

The term “(C₁-C₆)-alkyl” means hydrocarbon radicals whose carbon chain is straight-chain or branched and comprises 1 to 6 carbon atoms, for example methyl, ethyl, propyl, isopropyl, butyl, isobutyl, tertiary butyl, pentyl, isopentyl, neopentyl, hexyl, 2,3-dimethylbutane or neohexyl.

The term “—(CH₂)ₙ— in which n is the number zero, 1, 2 or 3” means when n equals zero a covalent bond, n equals 1 the methylene radical, n equals 2 the ethylene radical and n equals 3 propylene. The meanings of the term “(CH₂)ₘ— in which m is the number zero, 1, 2 or 3” are analogous to the term —(CH₂)ₙ—.

The term “—(C₂-C₄)-alkenylene” means hydrocarbon radicals whose carbon chain is straight-chain or branched and comprises 2 to 4 carbon atoms and, depending on the chain length, have 1 or 2 double bonds, for example ethenylene, propenylene, isopropenylene, isobutenylene or butenylene; the substituents on the double bond may, where the possibility exists in principle, have the E or Z orientation.

The term “—(C₂-C₆)-alkynylene” means hydrocarbon radicals whose carbon chain is straight-chain or branched and comprises 2 to 6 carbon atoms and, depending on the chain length, have 1 or 2 triple bonds, for example ethynylene, propenylene, isopropynylene, isobuthylynylene, butynylene, pentynylene or isomers of pentynylene or hexynylene or isomers of hexynylene.

The term “(C₃-C₆)-cycloalkyl” means radicals such as compounds which are derived from 3- to 6-membered monocycles such as cyclopropyl, cyclobutyl, cyclopentyl or cyclohexyl. The term “(C₅-C₇)-cycloalkyl” means radicals such as compounds which are derived from 5- to 7-membered monocycles such as cyclopentyl, cyclohexyl or cyloseptyl.

The term “—(C₆-C₁₄)-aryl” means aromatic carbon radicals having 6 to 14 carbon atoms in the ring. Examples of —(C₆-C₁₄)-aryl radicals are phenyl, naphthyl, 1-naphthyl, 2-naphthyl, anthryl or fluorenyl. Naphthyl radicals and, in particular, phenyl radicals are preferred aryl radicals.

The term “Het ring” means ring systems having 4 to 15 carbon atoms which are present in one, two or three ring systems which are connected together and which comprise one, two, three or four identical or different heteroatoms from the series oxygen, nitrogen or sulfur. Example of these rings systems are the radicals acridinyl, azepinyl, azetidinyl, aziridinyl, benzimidazalinyl, benzimidazolyl, benzofuranyl, benzothiofuranyl, benzothiophenyl, benzoxazolyl, benzthiazolyl, benztriazolyl, benztetrazolyl, benzisoxazolyl, benzisothiazolyl, carbazolyl, 4aH-carbazolyl, carbolinyl, quinazolinyl, quinolinyl, 4H-quinolizinyl, quinoxalinyl, quinuclidinyl, chromanyl, chromenyl, cinnolinyl, decahydroquinolinyl, dibenzofuranyl, dibenzothiophenyl, dihydrofuran[2,3-b]-tetrahydrofuranyl, dihydrofuranyl, dioxolyl, dioxanyl, 2H, 6H-1,5,2-dithiazinyl, furanyl, furazanyl, imidazolidinyl, imidazolinyl, imidazolyl, 1H-indazolyl, indolinyl, indolizinyl, indolyl, 3H-indolyl, isobenzofuranyl, isochromanyl, isoindazolyl, isoindolinyl, isoindolyl, isoquinolinyl (benzimidazolyl), isothiazolidinyl, 2-isothiazolinyl, isothiazolyl, isoxazolyl, isoxazolidinyl, 2-isoxazolinyl, morpholinyl, naphthyridinyl, octahydroisoquinolinyl, oxadiazolyl, 1,2,3-oxadiazolyl, 1,2,4-oxadiazolyl, 1,2,5-oxadiazolyl, 1,3,4-oxadiazolyl, oxazolidinyl, oxazolyl, oxazolidinyl, oxothiolanyl, pyrimidinyl, phenanthridinyl, phenanthrolinyl, phenazinyl, phenothiazinyl, phenoxathiinyl, phenoxazinyl, phthalazinyl, piperazinyl, piperidinyl, pteridinyl, purynyl, pyranyl, pyrazinyl, pyroazolidinyl, pyrazolinyl, pyrazolyl, pyridazinyl, pyridooxazolyl, pyridoimidazolyl, pyridothiazolyl, pyridothiophenyl, pyridinyl, pyridyl, pyrimidinyl, pyrrolidinyl, pyrrolinyl, 2H-pyrrolyl, pyrrolyl, tetrahydrofuranyl, tetrahydroisoquinolinyl, tetrahydroquinolinyl, tetrahydropyridinyl, 6H-1,2,5-thiadiazinyl, 1,2,3-thiadiazolyl, 1,2,4-thiadiazolyl, 1,2,5-thiadiazolyl, 1,3,4-thiadiazolyl, thianthrenyl, thiazolyl, thienyl, thienothiazolyl, thienooxazolyl, thienoimidazolyl, thiomorpholinyl, thiophenyl, triazinyl, 1,2,3-triazolyl, 1,2,3-triazolyl, 1,2,4-triazolyl, 1,2,5-triazolyl, 1,3,4-triazolyl and xanthenyl. Preferred Het rings are the radicals benzofuranyl, benzimidazolyl, benzoxazolyl, benzothiazolyl, benzothiophenyl, 1,3-benzodioxolyl, quinazolinyl, quinolinyl, quinoxalinyl, chromanyl, cinnolinyl, furanyl; such as 2-furanyl and 3-furanyl; imidazolyl, indolyl, indazolyl, isoquinolinyl, isochromanyl, isoindolyl, isothiazolyl, isoxazolyl, oxazolyl, phthalazinyl, pteridinyl, pyrazinyl, pyrazolyl, pyridazinyl, pyridoimidazolyl, pyridopyridinyl, pyridopyrimidinyl, pyridyl; such as 2-pyridyl, 3-pyridyl or 4-pyridyl; pyrimidinyl, pyrrolyl; such as 2-pyrrolyl and 3-pyrrolyl; purinyl, thiazolyl, tetrazolyl or thienyl; such as 2-thienyl and 3-thienyl.

The term “R1 and R2 form together with the carbon atoms to which they are bonded a 5-, 6- or 7-membered Het ring” means compounds which are derived for example from the following compounds such as dioxane, furan, imidazole, imidazoline, imidazolidine, isothiazole, isothiazolidine, isothiazoline, isoxazole, isoxazoline, isoxazolidine, 2-isoxazolines, morpholine, piperazines, 1,2-oxazine, 1,3-oxazine, 1,4-oxazine, oxazole, oxazolidine, oxazolidone, piperazine, piperidine, pyran, pyrazine, pyrazole, pyrazoline, pyrazolidine, pyridazine, pyridine, pyrimidine, pyrrole, pyrrolidine, pyrrolidinone, pyrroline, tetrahydrofuran, tetrahydropyran, tetrahydropyridine, tetrazine, tetrazole, thiadiazine, 1,2-thiazine, 1,3-thiazine, 1,4-thiazine, 1,3-thiazole, thiazole, thiazolidine, thiazoline, thiophene, thietane, thiomorpholine, thiopyran, 1,2,3-triazine, 1,2,4-triazine, 1,3,5-triazine, 1,2,3-triazole or 1,2,4-triazole.

The term “halogen” means fluorine, chlorine, bromine or iodine.

The invention further relates to a process for preparing the compound of the formula I and/or a stereoisomeric form of the compound of the formula I and/or a physiologically tolerated salt of the compound of the formula I, which comprises
a) converting a compound of the formula III

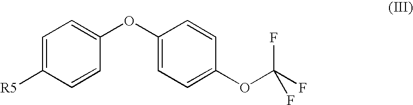

in which R5 is hydrogen atom, NH₂, Li, Mg. SH, S—CH₃, Cl, Br, I or Si—(CH₃)₃,
into a compound of the formula IV,

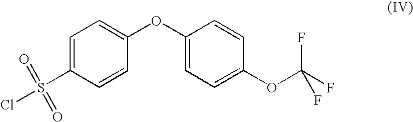

and reacting with a compound of the formula V,

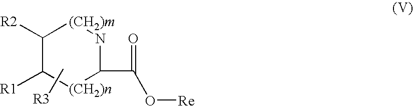

in which R1, R2, R3, m and n are as defined in formula I, and Re is a hydrogen atom or an ester protective group,
in the presence of a base or after silylation with a suitable silylating agent to give a compound of the formula VI,

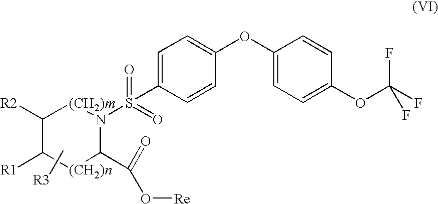

in which R1, R2, R3, m and n are as defined above, and
b) in the case where Re=ester reacting a compound of the formula VI prepared as in a) with an alkali metal hydroxide solution such as NaOH or LiOH and subsequent acid treatment to give the compound of the formula I, or reacting said ester by treatment with mineral acids such as hydrochloric acid to give the carboxylic acid of the formula VII,

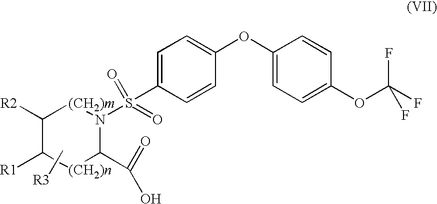

and subsequently converting the latter into the hydroxamic acid in which X═NH—OH, of the formula I,
c) fractionating a compound of the formula I which has been prepared by process a) or b) and which, because of its chemical structure, occurs in enantiomeric forms into the pure enantiomers by salt formation with enantiopure acids or bases, chromatography on chiral stationary phases or derivatization using chiral enantiopure compounds such as amino acids, separation of the diastereomers obtained in this way, and elimination of the chiral auxiliary groups, or
d) either isolating the compound of the formula I which has been prepared by processes b) or c) in free form or, in the case where acidic or basic groups are present, converting it into physiologically tolerated salts.

Compounds of the type of formula VI to VII represent only exemplary compounds; it is possible to mention instead of the six-membered ring corresponding to the formula I also four-membered rings, five-membered rings and seven-membered rings.

Compounds of the type of the formula V can be prepared by known methods.

For example, compounds with n equal to 1 and m equal to 0 (methanoprolines) can be prepared by several known processes. A recent synthesis is described for example in Tetrahedron 53, 14773-92 (1997).

For example, the basic bicyclic structures of the formula V with n=1 and m=1 according to formula I can be prepared by hydrogenation of the isoquinoline-1-carboxylic acid or suitable derivatives of the isoquinoline-1-carboxylic acid, such as the methyl or ethyl ester. This hydrogenation is described for example in U.S. Pat. No. 5,430,023, U.S. Pat. No. 5,726,159 and EP 643073.

It is likewise possible to employ 1,2,3,4-tetrahydroisoquinoline-1-carboxylic acid and derivatives thereof for the preparation of these compounds by hydrogenation. This process has the advantage that it is possible to employ a wide range of processes for synthesizing the 1,2,3,4-tetrahydroisoquinoline-1-carboxylic acids. Particularly well known and broadly applicable are, for example, Pictet-Spengler type cyclizations as described in U.S. Pat. No. 4,902,695. It is possible by such processes to obtain for example—depending on the nature of the starting materials employed—substituted compounds, i.e. compounds in which the substituents R1, R2 and R3 are not H atoms. A new example of ring-substituted compounds is to be found in WO 2003/041641.

Further methods for preparing the basic cyclic structures are possible for example by free-radical cyclization reactions and are described in Tetrahedron 48, 4659-76 (1992).

Other processes can be employed for synthesizing compounds of the type V if n is 1 and m is 0, Syntheses are described for example in Tetrahedron 55, 8025 (1999), Tetrahedron Lett. 24, 5339 (1983) and in the published specifications DE 3322530 and DE 3211676. It may under certain conditions be worthwhile to employ compounds of the type V in N-protected state. For example, compounds protected in this way can be purified better than the free imino acids, and they can likewise in some circumstances be employed better for preparing the enantiomerically or diastereomerically pure compounds. Groups which can be employed as protective groups for the imino group are those described in “Protective Groups in Organic Synthesis”, T. H. Greene, P. G. M. Wuts, Wiley-Interscience, 1999. Preferred amino or imino protective groups are, for example, Z, Boc, Fmoc, Aloc, acetyl, trifluoroacetyl, benzoyl, benzyl and the like.

The reactions take place for example as described in WO 97/18194. The reaction according to process step a) takes place in the presence of a base such as KOH, NaOH, LiOH, N-methylmorpholine (NMM), N-ethylmorpholine (NEM), triethylamine (TEA), diisopropylethylamine (DIPEA), pyridine, collidine, imidazole or sodium carbonate, in solvents such as tetrahydrofuran (THF), dimethylformamide (DMF), dimethylacetamide, dioxane, acetonitrile, toluene, chloroform or methylene chloride, or else in the presence of water. In the case where the reaction is carried out with use of silylating agents, for example N,O-bis(trimethylsilyl)acetamide (BSA) or N,O-bis(trimethylsilyl)trifluoro-acetamide (BSTFA) is employed for silylating the imino acid in order then to carry out the sulfonamide formation.

Modifications in the side chain F means that, for example, a nitro group is hydrogenated with the metal catalyst Pd/C or reacted with SnCl₂ or Zn under standard conditions, and the resulting amino group can subsequently be modified further, for example by reaction with carbonyl chlorides, sulfonyl chlorides, chloroformic esters, isocyanates, isothiocyanates or other reactive or activatable reagents, in order to obtain the precursors of the compounds of the invention of the formula I. It is often beneficial in this case for Re in compound III to be an ester, because side reactions must be expected in the case of the unprotected carboxylic acid.

In process step c), the compound of the formula I is, if it occurs as mixture of diastereomers or enantiomers or results as mixtures thereof in the chosen synthesis, is separated into the pure stereoisomers, either by chromatography on an optionally chiral support material or, if the racemic compound of the formula I is capable of salt formation, by fractional crystallization of the diastereomeric salts formed with an optically active base or acid as auxiliary. Examples of suitable chiral stationary phases for thin-layer or column chromatographic separation of enantiomers are modified silica gel supports (called Pirkle phases) and high molecular weight carbohydrates such as triacetylcellulose. For analytical purposes, gas chromatographic methods on chiral stationary phases can also be used after appropriate derivatization known to the skilled worker. To separate enantiomers of the racemic carboxylic acids, diastereomeric salts differing in solubility are formed using an optically active, usually commercially available, base such as (−)-nicotine, (+)- and (−)-phenylethylamine, quinine bases, L-lysine or L- and D-arginine, the less soluble component is isolated as solid, the more soluble diastereomer is deposited from the mother liquor, and the pure enantiomers are obtained from the diastereomeric salts obtained in this way. It is possible in the same way in principle to convert the racemic compounds of the formula I containing a basic group such as an amino group with optically active acids such as (+)-camphor-10-sulfonic acid, D- and L-tartaric acid, D- and L-lactic acid and (+) and (−)-mandelic acid into the pure enantiomers. Chiral compounds containing alcohol or amine functions can also converted with appropriately activated or, where appropriate, N-protected enantiopure amino acids into the corresponding esters or amides, or conversely chiral carboxylic acids can be converted with carboxyl-protected enantiopure amino acids into the amides or with enantiopure hydroxy carboxylic acids such as lactic acid into the corresponding chiral esters. The chirality of the amino acid or alcohol residue introduced in enantiopure form can then be utilized for separating the isomers by carrying out a separation of the diastereomers which are now present by crystallization or chromatography on suitable stationary phases, and then eliminating the included chiral moiety by suitable methods.

A further possibility with some of the compounds of the invention is to employ diastereomerically or enantiomerically pure starting materials to prepare the structures. It is thus possible where appropriate also to employ other or simplified processes for purifying the final products. These starting materials have previously been prepared enantiomerically or diastereomerically pure by processes known from the literature. For example, it is possible in the process for preparing the decahydroisoquinoline-1-carboxylic acid either to employ the isoquinoline-1-carboxylic acid directly, as stated and quoted above. Owing to the fact that 3 stereo centers are present, in this case a maximum of 8 stereoisomers (4 enantiomeric pairs of diastereomers) can be formed. However, certain stereoisomers are highly preferred through the manner of preparation, for example hydrogenation. It thus ought to be possible, as described in the literature, to achieve for example a strong preference for hydrogen addition onto the positions of the ring junction by suitable choice of the hydrogenation conditions (catalyst, pressure, solvent, temperature). It is thus possible under the stated conditions to achieve formation of rings with a cis junction. The position of the carboxylic acid would then remain to be determined; the number of possible stereoisomers would already be restricted to 4. Owing to the nature of the hydrogenation mechanism, it is possible particularly easily for the hydrogens to undergo addition on the same side as the bridge head hydrogens, i.e. a further restriction of the possibility of isomer formation is to be expected thereby. Thus, in the most favorable case, it could be assumed that only one pair of enantiomers will be formed. It should then be possible to fractionate the latter into the enantiomers by the abovementioned methods. However, it must also be assumed in these conjectures that complete stereoselection never takes place; on the contrary that larger or smaller amounts of the other isomers are also almost always formed and can be detected by suitable methods even in tiny amounts. In the case where enantiopure 1,2,3,4-tetrahydroisoquinoline-1-carboxylic acid derivatives are employed, it would be expected that, with reaction conditions which are identical or similar to the hydrogenation of the isoquinoline-1-carboxylic acid, analogous conjectures apply and again only preferred stereoisomers are formed in large amounts; there ought in said case to be a strong preference for a single enantiomer because in the hydrogenation process under analogous conditions which lead to the cis ring junction in the hydrogenation of the isoquinoline-1-carboxylic acid, again only addition of the H atoms from one side is possible likewise in this case, and thus analogous products are formed. The identity of the structures can be established by suitable 2D NMR experiments, X-ray methods such as, for example, cocrystallization or others, and comparative analysis or chemical derivatization and suitable analysis or chemical derivatization which leads to known and described isomers.

Another possibility for synthesizing enantiomerically or diastereomerically pure compounds is to employ suitable chirally substituted starting materials in order to achieve through the chiral substituents an induction of chirality at other chirality centers. For example, chiral glyoxylic esters might be employed in Pictet-Spengler cyclizations in order to obtain chiral Tic derivatives and then to hydrogenate the latter as already mentioned above.

Acidic or basic products of the compound of the formula I may exist in the form of their salts or in free form. Preference is given to pharmacologically acceptable salts, for example alkali metal or alkaline earth metal salts, or hydrochlorides, hydrobromides, sulfates, hemisulfates, all possible phosphates, and salts of amino acids, natural bases or carboxylic acids. The preparation of physiologically tolerated salts from compounds of the formula I which are capable of salt formation, including their stereoisomeric forms, in process step d) takes place in a manner known per se. The compounds of the formula I form stable alkali metal, alkaline earth metal or, where appropriate, substituted ammonium salts with basic reagents such as hydroxides, carbonates, bicarbonates, alcoholates, and ammonia or organic bases, for example trimethylamine or triethylamine, ethanolamine, diethanolamine or triethanolamine, trometamol or else basic amino acids, for example lysine, ornithine or arginine. If the compounds of the formula I have basic groups, stable acid addition salts can also be prepared with strong acids. Suitable for this purpose are both inorganic and organic acids such as hydrochloric, hydrobromic, sulfuric, hemisulfuric, phosphoric, methanesulfonic, benzenesulfonic, p-toluenesulfonic, 4-bromobenzenesulfonic, cyclohexylamidosulfonic, trifluoromethylsulfonic, 2-hydroxyethanesulfonic, acetic, oxalic, tartaric, succinic, glycerophosphoric, lactic, malic, adipic, citric, fumaric, maleic, gluconic, glucuronic, palmitic, or trifluoroacetic acid.

The invention also relates to novel intermediates of the formula III

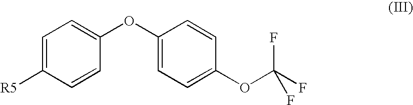

in which R5 is hydrogen atom, NH₂, Li, Mg, SH, S—CH₃, Cl, Br, I, Si—(CH₃)₃, SO₂—Cl, SO₂—Br, SO₂—Y, in which Y is a radical which can easily be eliminated, such as an active ester O—R_{y}, where Ry is ortho- or para-nitrophenyl, 2,4-dinitrophenyl, or pentafluorophenyl, or Y is a heterocycle such as imidazole, benzimidazole or benzotriazole, in which case the linkage takes place via the nitrogen of the heterocycle.

Active ester processes are described for example in J. Org. Chem. 45, 547 (1980), Indian J. Chem. 25, 1273 (1986), J. Org. Chem. 57, 190 (1992), Nippon Kagaku Kaishi 4, 631-6 (1976) and Tetrahedron Lett. 28, 2115 (1987).

A preferred variant for preparing the compounds of the formula III in which R5 is SO₂—Cl, SO₂—Br or SO₃H starts from the appropriately substituted diaryl ether. The preparation of these arylsulfonyl chlorides and -sulfonic acids is disclosed in the literature and can take place by various processes.

A frequently used synthesis starts from the compounds of the formula VIII

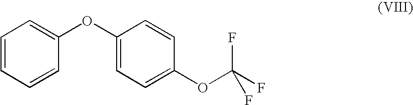

which can be converted by reaction with chlorosulfonic acid into the arylsulfonic acid or, on use of an excess of chlorosulfonic acid, also directly into the arylsulfonyl chlorides. The position of the radical to be introduced is in this case dependent on the directing influence of other substituents. Phenoxy substituents, as in the present case, direct entering substituents such as the sulfonic acid residue into the desired para position. However, care must be taken that the reaction conditions are maintained because multiple sulfonations or other undesired side reactions may occur in some circumstances. If the sulfonic acid is initially prepared by said process, conversion into the sulfonyl chloride is possible by many different methods. Those employed successfully are oxalyl chloride, phosphorus oxychloride, phosphorus pentachloride, thionyl chloride and also other methods for chlorination. Methods for synthesis via chlorosulfonic acid are described in many sources, for example in Org. Synth. I, 8 and 85 (1941). Further known methods can be used to introduce the sulfonic acid residue into the compound of the formula VIII. Examples employed are:
concentrated sulfuric acid (Recl. Tray. Chim. Pays-Bas 107, 418 (1988), silylated sulfuric acid (Bull. Soc. Chim. Fr. 1980, p. 195), J. Am. Chem. Soc. 71, 1593 (1949)), sulfur trioxide ((Recl. Tray. Chim. Pays-Bas 111, 215 (1992), mixtures of sulfur trioxide and sulfur dioxide (J. Prakt. Chem. 22, 290 (1963)), mixtures of sulfur trioxide and concentrated sulfuric acid (J. Prakt. Chem. 93, 183 (1916)).

Another frequently used method starts from arylamines. These are initially converted in a diazotization reaction into the diazo compound, for example by reaction by sodium nitrite in concentrated aqueous hydrochloric acid, and subsequently converted with copper catalysis, for example with CuCl or CuCl₂, into the sulfonyl chlorides with SO₂, preferably in acetic acid. See, for example: Bioorg. Med. Chem. Lett. 9, 1251 (1999), J. Med. Chem. 27, 1740 (1984), Org. Synth. 60, 121 (1981), Chem. Ber. 90, 841 (1957), Org. Synth. VII, 508 (1990).

Another method starts from compounds of the formula IX

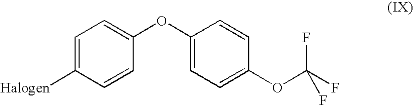

in which halogen is Cl, Br or I. These are converted with alkyllithium, for example n-BuLi, (Bu stands for butyl), into the lithiated aryls. These are subsequently converted by reaction with SO₃-amine adducts (such as trimethylamine) into the sulfonic acid. Reaction with SO₂ and NCS or SO₂ and SOCl₂ is also described, resulting directly in the chlorinated derivatives. These reactions are described for example in J. Org. Chem. 61, 1530 (1996), J. Chem. Soc., Perkin I 13, 1583 (1996) and Synthesis 1986, p. 852. Grignard-like reactions are likewise described: Chem. Ber. 128, 575 (1995).

Sulfonyl chlorides can likewise be prepared by oxidation of arylthiols with subsequent chlorination: Chem. Lett. 8, 1483 (1992).

Silylated phenoxyphenyls can be converted with silylated chlorosulfonic acid under phase-transfer conditions into the sulfonic acids (Synthesis 11, 1593 (1998).

The preparation of compounds of the formula III in which R5 is SO₃ can be carried out in particular by two different processes.

The preferred process in this connection is the diaryl ether synthesis employing one building block which already has a trifluoromethoxy group. This may preferably be for example either the 4-trifluoromethoxybenzenes or one of its related derivatives, or else the 4 substituted 4-trifluoromethoxyphenyls which comprise a replaceable F, Cl, Br, I. The reactant employed in the first case is, for example, a halobenzene or phenol for the second case. Other replaceable substituents are also possible, depending on the synthesis used and as described for example in recent syntheses. This starting material can either be prepared by known methods or can be purchased. Diaryl ether syntheses are described widely, a recent synthesis for example in Org. Lett. 6, 913 (2004). Review articles indicate a large number of methods, e.g. in: Tetrahedron 56, 5045 (2000); J. Heterocycl. Chem. 36, 1453 (99); Org. Lett. 5, 3799 (2003); Synlett 11, 1734 (2003); Organic Chemistry 2002, p. 1-8; J. Am. Chem. Soc. 119, 10539 (1997).

It is likewise possible also to employ a suitably 4-substituted benzenesulfonic acid derivative such as, for example, 4-bromobenzenesulfonyl chloride. This is reacted with at least 2 equivalents of 4-trifluoromethoxyphenol under the described conditions of the aryl ether synthesis and affords the corresponding sulfonic acid aryl ester of the diaryl ether. It is then necessary for a preferably basic cleavage of the sulfonic ester to the sulfonic acid to take place before the acid chloride of the formula IV is obtained by chlorination.

A further process which can be used can be regarded as constructing the trifluoromethoxy side chain from the corresponding 4-phenoxyphenol. However—owing to the particular properties of the trifluoromethoxy group—only a few specific processes are known because there is only provisional analogy with simple alkyl ethers. 4-Phenoxyphenol can be deprotonated with various strong bases. A nucleophilic substitution reaction is then carried out with dibromodifluoromethane. The resulting bromodifluoromethoxyphenoxyphenol can then be fluorinated using mild fluorination methods, for example with pyridine-HF (U.S. Pat. No. 4,782,094 and EP 0257415). The further reactions to give the compound of the formula III can be carried out as described above.

The compounds of the formula III can be employed for synthesizing pharmacologically active compounds. These often have an activity similar to analogous nonfluorinated derivatives. However, many different properties of a compound need to be adjusted and optimized in the drug-finding process. The uptake, disposition, metabolism and excretion are, besides the biological activity, of decisive importance so that early testing for these properties is very important in the drug-finding process, and negative properties here may lead to early termination of the profiling of active substances.

It has now surprisingly been found that compounds which have as contributory structure the compound of the formula IV have distinctly better pharmacokinetic properties than structurally similar compounds having unsubstituted alkyl ether or alkyl fluoride as side chains.

The invention also relates to medicaments having an effective content of at least one compound of the formula I and/or of a physiologically tolerated salt of the compound of the formula I and/or an optionally stereoisomeric form of the compound of the formula I, together with a pharmaceutically suitable and physiologically tolerated carrier, additive and/or other active substances and excipients.

Because of the pharmacological properties, the compounds of the invention are suitable for the selective prophylaxis and therapy of all disorders in the progression of which an enhanced activity of metalloproteinases are involved. These include degenerative joint disorders such as osteoarthroses, spondylosis, chondrolysis after joint trauma or prolonged joint immobilization after meniscus or patellar injuries or ligament tears. They also include connective tissue disorders such as collagenoses, periodontal disorders, wound-healing disturbances and chronic disorders of the locomotor system such as inflammatory, immunologically or metabolism-related acute and chronic arthritides, arthropathies, myalgias and disturbances of bone metabolism. The compounds of the formula I are also suitable for the treatment of ulceration, atherosclerosis and stenoses. The compounds of the formula I are furthermore suitable for the treatment of inflammations, cancers, tumor metastasis, cachexia, anorexia, heart failure and septic shock. The compounds are likewise suitable for the prophylaxis of myocardial and cerebral infarctions.

The medicaments of the invention can be administered by oral, inhalational, rectal or transdermal administration or by subcutaneous, intraarticular, intraperitoneal or intravenous injection. Oral administration is preferred.

The invention also relates to a process for producing a medicament which comprises converting at least one compound of the formula I with a pharmaceutically suitable and physiologically tolerated carrier and, where appropriate, further suitable active substances, additives or excipients into a suitable dosage form.

Examples of suitable solid or pharmaceutical preparations are granules, powders, coated tablets, tablets, (micro)capsules, suppositories, syrups, oral solutions, suspensions, emulsions, drops or injectable solutions, and products with protracted release of active substance, in the production of which conventional aids such as carriers, disintegrants, binders, coating agents, swelling agents, glidants or lubricants, flavorings, sweeteners and solubilizers are used. Excipients which are frequently used and may be mentioned are magnesium carbonate, titanium dioxide, lactose, mannitol and other sugars, talc, milk protein, gelatin, starch, cellulose and its derivatives, animal and vegetable oils such as fish liver oil, sunflower, peanut or sesame oil, polyethylene glycol and solvents such as, for example, sterile water and monohydric or polyhydric alcohols such as glycerol.

The pharmaceutical products are preferably produced and administered in dosage units, each unit comprising as active ingredient a particular dose of the compound of the invention of the formula I. In the case of solid dosage units such as tablets, capsules, coated tablets or suppositories, this dose can be up to about 1000 mg, but preferably about 50 to 300 mg, and in the case of solutions for injection in ampoule form up to about 300 mg, but preferably about 10 to 100 mg.

The daily doses indicated for the treatment of an adult patient weighing about 70 kg are from about 2 mg to 1000 mg of active substance, preferably about 50 mg to 500 mg, depending on the activity of the compound of the formula I. However, in some circumstances, higher or lower daily doses may also be appropriate. The daily dose may be administered both by administration once a day in the form of a single dosage unit or else a plurality of smaller dosage units, and by administration more than once a day in divided doses at defined intervals.

Final products are usually determined by mass spectroscopic methods (FAB-, ESI-MS) and ¹H NMR (400 MHz, in DMSO-D6), with the main peak or the two main peaks being indicated in each case. Temperatures are stated in degrees Celsius, RT means room temperature (21° C. to 24° C.). Abbreviations used are either explained or correspond to the usual conventions.

The invention is explained in detail below by means of examples. The general methods can be used to synthesize the compounds of the formula I.

### EXAMPLES

### General Method 1

### Sulfonamide from Sulfonyl Chloride and Carboxylic Acid

The carboxylic acid (6.45 mmol) was dissolved in 20 ml of dimethylformamide (DMF) and, at 0° C., 3 equivalents of a 3N NaOH solution (6.45 ml) were added. After 10 min a solution of the arylsulfonyl chloride (1.1 equivalents, 7.1 mmol) in 10 to 15 ml DMF was slowly added dropwise and, after room temperature (RT) was reached, the mixture was stirred at temperatures between 20° C. and 80° C. for a maximum of 12 hours (h). The exact time is ascertained according to the conversion which has taken place, which was established by mass spectroscopy. The solvent was then removed under reduced pressure. An aqueous workup then took place (extraction with 1N HCl and saturated NaCl solution, drying of the organic phase such as ethyl acetate, methylene chloride or chloroform with magnesium sulfate or sodium sulfate, then concentration). The crude product was either directly reacted further or purified by chromatography.

### General Method 2

### Sulfonamide from Sulfonyl Chloride and Carboxylic Acid

The carboxylic acid was dissolved in 0.5-2 molar NaOH, possibly with addition of 10-50% tetrahydrofuran (THF) or DMF. Acid chloride (1-1.2 equivalents, preferably 1.1) was dissolved in THF (concentration 0.05 to 1M) and slowly added dropwise. 2N NaOH was added automatically in an autotitrator at RT to keep the pH constant. Adjusted pH: 8 to 12, preferably 9 to 11. After the reaction is complete, evident from no further NaOH consumption, the organic cosolvent was removed in a rotary evaporator, and the aqueous solution or suspension was mixed with ethyl acetate and acidified with 1N HCl. After removal of the organic phase and renewed extraction of the aqueous phase with ethyl acetate, the organic phases were combined, dried over sodium sulfate and then the solvent was removed under reduced pressure. The crude product was either directly reacted further or purified by chromatography.

### General Method 3

### Sulfonamide from Sulfonyl Chloride and Carboxylic Acid

8 mmol of the imino acid were dissolved or suspended in 30 ml of acetonitrile. At RT and under inert gas (N₂), 2.3 g (9 mmol) of BSTFA (bis(trimethylsilyl)trifluoroacetamide) were added, and the mixture was heated under reflux for 2 h. 2.84 g (9 mmol) of the sulfonyl chloride dissolved in 30 ml of acetonitrile were added to this solution, and the mixture was again heated under reflux conditions for 3 h. After the reaction mixture had cooled, aqueous 1N HCl was added and stirred for 1 h, the solvent was removed under reduced pressure in a rotary evaporator, and then ethyl acetate or chloroform was added, the organic phase was separated off and extracted with saturated NaCl solution, dried over sodium sulfate and concentrated under reduced pressure. Depending on the purity of the reaction product, it could be directly reacted further or require previous chromatography on silica gel.

### General Method 4

### Preparation of the Hydroxamic Acid from Carboxylic Acid Via Chloroformate Activation

The sulfonated carboxylic acid was dissolved in 10 ml of DMF and, at 0° C., 1.1 equivalents of ethyl chloroformate, 2.2 equivalents of N-ethylmorpholine and—after a preactivation time of 30 min to 1 h-3 equivalents of trimethylsilylhydroxylamine were added. After the mixture had been heated at 80° C. for at least 4 h, the solvent was removed under reduced pressure and the crude product was purified by chromatographic methods.

### General Method 5

### Preparation of the Hydroxamic Acid Through Via the Corresponding Carbonyl Chloride

The sulfonated carboxylic acid was introduced into dry chloroform (ethanol-free) (about 5 ml for 0.5 mmol) and, at RT, 3 equivalents of oxalyl chloride were added. The mixture was then heated at 45° C. for about 30 min. To check the chloride formation, a small sample was taken from the reaction flask and mixed with a little benzylamine in THF. Complete reaction was evident from quantitative benzylamide formation, the carboxylic acid no longer being detectable (checked by HPLC-MS). It is necessary where appropriate to heat for a longer time or heat under reflux conditions. The solvent was then removed by distillation under reduced pressure, and the residue was taken up in dry toluene and again evaporated to dryness several times. The acid chloride was then taken up in chloroform (10 ml per 0.5 mmol) and, at RT, 3 equivalents of O-trimethylsilylhydroxylamine were added. After a reaction time of at least 30 min (reaction checked by HPLC-MS), the reaction mixture was evaporated under reduced pressure and the residue was purified by direct chromatography.

### Example 1

### 4-Trifluoromethoxyphenoxybenzene

4-Trifluoromethoxybromobenzene (10 g, 41.5 mmol), phenol (3.9 g, 41.5 mmol), potassium carbonate (8.03 g, 58 mmol) and copper-1 chloride (103 mg, 1.04 mmol) were mixed in dry DMF. The mixture was stirred at 150° C. under argon for 28 h. The reaction mixture was then concentrated in a rotary evaporator, and the residue was taken up in ethyl acetate and mixed with 10% strength sodium carbonate solution and solid sodium thiosulfate. Fine solid constituents were removed by passing both phases through a frit with kieselguhr and subsequently separating, and the aqueous phase was extracted twice more with ethyl acetate. The combined organic phases were dried over sodium sulfate and evaporated under reduced pressure. Precursor components and by-products were removed by carrying out a flash chromatography on silica gel (eluent: n-heptane-ethyl acetate 10:1). Product fractions were combined.

Yield: 2.5 g, 24% of theory; ¹H NMR: 7.09 (m, 4H); 7.20 (m, 1H); 7.42 (m, 4H). MS: 255.2 (ES+)

### Example 2

### 4-(4-Trifluoromethoxyphenoxy)benzenesulfonyl chloride

The product from Example 1 (2.4 g, 9.44 mmol) was dissolved in 25 ml of dichloromethane; while cooling with ice-water, a solution of chlorosulfonic acid in 5 ml of dichloromethane (0.84 g, 7.2 mmol) was slowly added dropwise, and the mixture was stirred at RT for 2.5 h. Further dichloromethane was added, and the mixture was extracted with a little water. A fine solid was removed by filtration through kieselguhr. The organic phase was separated off and dried over sodium sulfate and, after removal of the desiccant by filtration, evaporated. Direct reaction further was carried out by dissolving in 25 ml of dichloromethane, slowly adding oxalyl chloride (0.823 ml, 1.2 g, 9.44 mmol) dropwise, adding 0.5 ml of DMF and stirring at 40° C. for 1 h, storing at 4° C. overnight and, the following day after a check of the reaction by LC-MS and further addition of 0.5 ml of oxalyl chloride, renewed stirring at 40° C. for 2 h. The reaction mixture was poured onto ice and extracted with ethyl acetate. The organic phase was washed with saturated NaCl solution and then separated off and dried over sodium sulfate. Removal of the desiccant by filtration was followed by addition of toluene and evaporation under reduced pressure.

Yield: 4.56 g (>100%, contains salts) ¹H-NMR (in CDCl₃): 6.82; 6.91; 7.12; 7.57 (4 m, 8H) MS: 352.0 (ES+)

Hydrolysis (water in acetonitrile) affords pure sulfonic acid.

¹H NMR (in DMSO-D6): 6.99; 7.13; 7.40; 7.54 (4 m (“d”), 8H) MS: 333.2 (ES−)

### Example 3

### 2-[4-[4-Trifluoromethoxyphenoxy)benzenesulfonyl]-1,2,3,4-tetrahydroisoquinoline-1-(N-hydroxy)carboxamide

Step 1: Sulfonamide Formation

Tetrahydroquinoline-1-carboxylic acid (502 mg, 2.84 mmol) was dissolved or suspended in 60 ml of acetonitrile. At RT and under inert gas (N₂), 1.85 g (9.07 mmol) of BSA (bis(trimethylsilyl)cetamide) were added, and the mixture was heated under reflux for 0.5 h. 1.0 g (2.84 mmol) of the compound from Example 2, dissolved in 10 ml of acetonitrile, was added to this solution, and the mixture was again heated under reflux conditions for 2 h. After the reaction mixture had cooled, aqueous 1N HCl was added, the mixture was stirred for 1 h, the solvent was removed under reduced pressure in a rotary evaporator, and then ethyl acetate was added, the organic phase was separated off and extracted with saturated NaCl solution, dried over sodium sulfate and concentrated under reduced pressure. The purity of the product was checked by LC-MS and the resulting crude product was then directly reacted further.

Step 2: Hydroxamate Synthesis

The compound from step 1 was dissolved in 40 ml of chloroform. Oxalyl chloride (1.585 g, 4.99 mmol, 1.093 ml) was then added dropwise over the course of 20 min, and the resulting reaction mixture was heated at 40-45° C. for 2 h. The solvent was then removed by distillation under reduced pressure, and the resulting oily residue was entrained with toluene to remove any oxalyl chloride residues or HCl and left under reduced pressure for 15 min. It was then again taken up in chloroform (40 ml) and, at RT, O-trimethylsilylhydroxylamine (0.41 g, 3.9 mmol) was added. After 2 hours, the solvent was removed under reduced pressure, and the residue was dissolved in a small amount of an acetonitrile-water-0.01% trifluoroacetic acid mixture for direct preparative RP-HPLC. Product fractions were combined, acetonitrile was removed under reduced pressure, and the remaining aqueous phase was freeze-dried.

Yield: 580 mg (39% of theory). ¹H-NMR: 2.7, 2.9 (2m, 2H); 3.6, 4.0 (2m, 2H) 5.21 (s, 1H); 7.2 (m, 8H); 7.45, 7.81 (dd, 4H); 9.0 (s, br, 1H); 11.1 (s, 1H) MS: 508.09 (ES+)

The compounds according to Examples 4 to 7 were synthesized in a manner analogous to the above description.

### Example 4

### 2-[4-(4-Trifluoromethoxyphenoxy)benzenesulfonyl]decahydroisoquinoline-1-(N-hydroxy)carboxamide

¹H NMR: 1.1-1.95 (4 m, 12H); 3.6 (overlapping with water; 2m, 2H); 4.1 (d, 1H); 7.12; 7.27; 7.48; 7.77 (2 dd, 8H); 8.9 (s, br., 1H); 10.9 (s, 1H) MS (ES+): 515.21

### Example 5

### 5-[4-(4-Trifluoromethoxyphenoxy)benzenesulfonyl]octahydrofuro[3,2-c]pyridine-4-carboxylic acid

¹H NMR: 1.5-2.1 (4 m, 4H); 2.55 (m, 1H); 3.25-3.85 (4 m, 4-5H, overlapping with water); 4.28 (d, 1H); 7.1 (d, 2H); 7.18; 7.27; 7.48; 7.80 (2 dd, 8H); 12.8 (s, 1H) MS (ES+): 488.07

### Example 6

### 5-[4-(4-Trifluoromethoxyphenoxy)benzenesulfonyl]octahydrofuro[3,2-c]pyridine-4-(N-hydroxy)carboxamide

MS (ES+): 503.10 (RT 1.442 min; YMC J′sphere ODS H80 20×2, 4μ; 30° C., 0 min 96% water, 0.05% TFA, 2.0 min-95% acetonitrile; 95% acetonitrile to 2.4 min; 4% acetonitrile 2.45 min; 1 ml/min. inj. vol. 0.4 μl)

### Example 7

### 2-[4-(4-Trifluoromethoxyphenoxy)benzenesulfonyl]decahydrobenzo[c]azepine-1-(N-hydroxy)carboxamide

¹H NMR: 1.1-2.3 (m, 14H); 3.6 (overlapping with water; 2m, 2H); 4.4 (d, 1H); 7.12; 7.25; 7.48; 7.79 (2 dd, 8H); 8.7 (s, br., 1H); 10.5 (s, 1H) MS (ES+): 529.25

Preparation of the Comparative Compounds

The comparative compounds in Table 2 were synthesized in the manner analogous to the above description.

The analogous sulfonyl chlorides having the methoxy and trifluoromethyl side chain are commercially available. Sulfonamide formation and hydroxamic acid formation is carried out in analogy to the above description. The ethoxy compound is prepared starting from 4-phenoxyphenol. Firstly the ethyl ether is introduced by standard processes of ether formation which are known to the skilled worker, via triflate activation, and subsequently reaction to give the sulfonyl chloride takes place in analogy to the above description. Sulfonamide formation and preparation of the hydroxamic acid takes place likewise in analogy to the above description.

### Example 8

### 2-[4-(4-Methoxyphenoxy)benzenesulfonyl]-1,2,3,4-tetrahydroisoquinoline-1-carboxylic acid

¹H NMR: 2.5-2.9 (m, 2H); 3.5-3.8 (m, 2H); 3.8 (s, split, 3H); 5.38 (s, 1H); 6.95-7.8 (m, 12H) MS (ES+): 439.11

### Example 9

### 2-[4-(4-Methoxyphenoxy)benzenesulfonyl]-1,2,3,4-tetrahydroisoquinoline-1-N-hydroxycarboxamide

¹H NMR: 2.68, 2.90, 3.50, 3.98 (4 m, 4H); 3.8 (s, 3H); 5.20 (s, 1H); 6.90-7.25 (m, 10H); 7.70 (d, 2H); 11.0 (s, 1H), MS (ES+): 454.12

### Example 10

### 2-[4-(4-Trifluorophenoxy)benzenesulfonyl]-1,2,3,4-tetrahydroisoquinoline-1-carboxylic acid

¹H NMR: 2.45-2.9 (m, 2H); 3.6-4.1 (2m, 2H); 5.45 (s, 1H); 6.1-7.9 (m, 12H); 9.0, 11.1 (2 s, 2H), MS (ES+): 493.06

### Pharmacological Examples

### Pharmacokinetic Measurements

### General Method

In each case 14 or 16 male C57/BL mice with an average weight of 20 to 28 g were used for the investigation, and divided into two groups. The animals had free access to feed and water. The substances were dissolved in PEG400/water 1:1 and administered orally by gavage in a concentration of about 7.5 mg per kg (equivalent to about 0.2 g per animal). In each case, 2 novel compounds and one comparative compound were previously dissolved separately, mixed and administered simultaneously (n-in-one study). Blood samples were taken after 0.25, 0.5, 1, 2, 4, 6, 8 and 24 h, and the substance concentration was determined quantitatively by HPLC-MS under standardized conditions as described below. The individual results were initially combined within the two groups and then the average was formed therefrom. The pharmacokinetic parameters are calculated using the noncompartmental model (extravascular input).

Quantification took place by HPLC-MS-MS. An HPLC system from Agilent (1100) was used, coupled to a PE-Sciex API 4000 (triple quadruple mass spectrometer). The column used was a ProdigyR 5μ ODS, flow rate 0.32 ml/ml, injected volumes 16 μl. Eluent: acetonitrile-0.002% ammonium formate. Detection took place in MS/MS mode (multiple reaction monitoring) focussed on Q1 and selective masses (fragment) filtration on Q3.

Workup of the plasma samples beforehand took place as follows: admixture of 25 μl of 60/40 acetonitrile/0.1% formate plus 25 μl of internal standard 5 μg/ml in the same solvent, plus 25 μl of blank plasma or sample plasma, plus 200 μl of acetonitrile. Mixing for 5 min was followed by centrifugation (3 min, 5000 g) and then pipetting of 200 μl into the measurement vessels.

Preparation and Determination of the Enzymatic Activity of the Catalytic Domain of Human Stromelysin (MMP-3) and of Neutrophil Collagenase (MMP-8).

The two enzymes stromelysin (MMP-3) and neutrophil collagenase (MMP-8) were prepared by the method of Ye et al. (Biochemistry; 31 (1992) pages 11231-11235). The enzymic activity or the effect of the enzyme inhibitor was measured by incubating 10 μl of enzyme solution with 10 μl of a 3% strength (v/v) buffered dimethyl sulfoxide solution which contained the enzyme inhibitor where appropriate, for 15 minutes. After addition of 10 μl of a 3% strength (v/v) aqueous dimethyl sulfoxide solution which contained 1 mmol/l of the substrate, the enzymic reaction was followed by fluorescence spectroscopy (328 nm (ex)/393 nm(em)).

The enzymic activity is presented as increase in extinction/minute. The IC₅₀ values listed in Table 1 were determined as the inhibitor concentrations leading in each case to 50% inhibition of the enzyme.

The buffer solution contained 0.05% Brij (Sigma, Deisenhofen, Germany) and 0.1 mol/l of Tris/HCl, 0.1 mol/l of NaCl, 0.01 mol/l of CaCl₂ and 0.1 mol/l of piperazine-N,N′-bis[2-ethanesulfonic acid] (pH=7.5).

The MMP-3 enzyme solution contained 2.3 μg/ml and the MMP-8 enzyme solution 0.6 μg/ml of one of the enzyme domains prepared by the method of Ye et al. The substrate solution contained 1 mmol/l of the fluorogenic substrate (7-methoxycoumarin-4-yl)acetyl-Pro-Leu-Gly-Leu-3-(2′,4′-dinitrophenyl)-L-2,3-diaminopropionyl-Ala-Arg-NH₂ (Bachem, Heidelberg, Germany).

Determination of the Enzymatic Activity of the Catalytic Domain of Human Collagenase-3 (MMP-13).

This protein was obtained as inactive proenzyme from INVITEK, Berlin, (catalogue No. 30 100 803). Activation of the proenzyme:

2 parts by volume of proenzyme were incubated with 1 part by volume of APMA solution at 37° C. for 1.5 hours. The APMA solution was prepared from a 10 mmol/l p-aminophenylmercuric acetate solution in 0.1 mmol/l NaOH by dilution with 3 parts by volume of Tris/HCl buffer pH 7.5 (see below). The pH was adjusted to between 7.0 and 7.5 by adding 1 mmol/l HCl. After activation of the enzyme it was diluted with the Tris/HCl buffer to a concentration of 1.67 μg/ml.

The enzymic activity was measured by incubating 10 μl of enzyme solution with 10 μl of a 3% strength (v/v) buffered dimethyl sulfoxide solution (reaction 1) for 15 minutes. The enzyme inhibitor activity was measured by incubating 10 μl of enzyme solution with 10 μl of a 3% strength (v/v) buffered dimethyl sulfoxide solution which contained the enzyme inhibitor (reaction 2).

The enzymic reaction both in the case of reaction 1 and in the case of reaction 2 was followed after addition of 10 μl of a 3% strength (v/v) aqueous dimethyl sulfoxide solution which contains 0.075 mmol/l of the substrate by fluorescence spectroscopy (328 nm (extinction)/393 nm (emission)).

The enzymic activity has been presented as increase in extinction/minute. The effect of the inhibitor was calculated as percentage inhibition by the following formula:

% inhibition=100−[(increase in extinction/minute in reaction 2)/(increase in extinction/minute in reaction 1)×100].

The IC₅₀, which is the concentration of inhibitor which is necessary for 50% inhibition of the enzymic activity, was determined graphically by plotting the percentage inhibitions at various inhibitor concentrations.

The buffer solution contained 0.05% Brij (Sigma, Deisenhofen, Germany) and 0.1 mol/l of Tris/HCl, 0.1 mol/l of NaCl, 0.01 mol/l of CaCl₂ (pH=7.5).

The enzyme solution contained 1.67 μg/ml of the enzyme domain. The substrate solution contained 0.075 mmol/l of the fluorogenic substrate (7-methoxycoumarin-4-yl)acetyl-Pro-Leu-Gly-Leu-3-(2′,4′-dinitrophenyl)-L-2,3-diaminopropionyl-Ala-Arg-NH₂ (Bachem, Heidelberg, Germany).

Examples of MMP inhibitors which surprisingly have particularly favorable properties with the described side chain: only the alkyl side chain was varied.

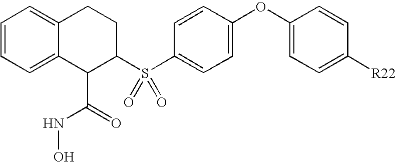

TABLE 1
Matrix metalloproteinase inhibitions (IC50 in nM) of the
compounds

| Example | R22 | MMP-3 | MMP-8 | MMP-13 |
|---|---|---|---|---|
| 3 | OCF₃ | 28 | 3.6 | 2.0 |
| 4 | OCF3 | 69 | 14 | 4 |
| 5 | OCF3 | 380 | 210 | 240 |
| 6 | OCF3 | 24 | 4 | 2 |
| 7 | OCF3 | 24 | 4 | 2 |

Comparative compound

| | | | | |
|---|---|---|---|---|
| 9 | OMe | 23 | 2.3 | 2.2 |

TABLE 2
Comparison of the PC data of the compound with the side chain of
the invention and the compound most similar thereto

| Example | R22 | Cmax (μg (ml) | t½ (h) | AUC (μg/ml * h) |
|---|---|---|---|---|
| 3 | OCF₃ | 1.35 | 6.6 | 2.23 |

Comparative compounds

| | | | | |
|---|---|---|---|---|
| | OEt | 0.33 | 4.7 | 0.43 |
| 9 | OMe | 0.45 | 1.5 | 0.25 |
| 10 | CF₃ | 0.24 | 1.8 | 0.16 |

Me stands for methyl radical; Et stands for ethyl radical

Cmax is the maximum plasma concentration reached at one of the sampling times. AUC “area under the curve”, time course of the decrease in concentration and Cmax determine the magnitude of the value.

The difference becomes particularly clear on comparison of the particularly relevant area under the curve, AUC. This value is a factor of about 5 better for the trifluoromethoxy compound of the invention than for the compound with a methoxy side chain.

1. A compound of the formula I 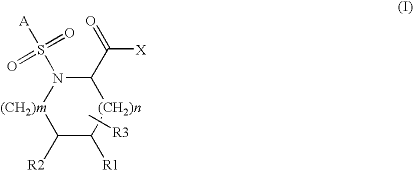
wherein
X is —OH or —NH—OH;
A is a radical of the formula II 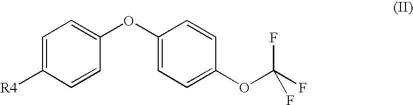
wherein R4 is the covalent bond connected to the S atom of formula I;
R3 is
hydrogen,
—(C₁-C₆)-alkyl that is unsubstituted or substituted once or twice by —(C₃-C₆)-cycloalkyl, —(C₂-C₄)-alkenylene, —(C₂-C₆)-alkynyl,
—C(O)—O—R8,
—O—R8,
-halogen,
—NO₂, or
—CN, or
R1 and R2 form together with the carbon atoms to which they are bonded form a phenyl ring, in which the ring is unsubstituted or substituted once by G;
R8 is hydrogen,
—(C₁-C₆)-alkyl that is unsubstituted or substituted once or twice by —(C₂-C₄)-alkenylene, or —(C₂-C₆)-alkynyl, or once to five times by fluorine;
G is halogen,
—(C₁-C₆)-alkyl that is unsubstituted or substituted once, twice or three times by halogen, —(C₂-C₄)-alkenylene, or —(C₂-C₆)-alkynyl,
—C(O)—O—R10,
—C(S)—O—R10,
—C(O)—NH—R11,
—C(S)—NH—R11,
—O—R12,
—C(O)—R10,
—S(O)ₚ—R12,
—NO₂,
—CN,
—N(R15)-R12, or
—SO₂—N(R12)-R16;
R10 is —(C₁-C₆)-alkyl that is unsubstituted or substituted once or twice by —(C₂-C₄)-alkenylene, or —(C₂-C₆)-alkynyl;
R11 is —(C₁-C₆)-alkyl;
R12 is hydrogen,
—(C₁-C₆)-alkyl that is unsubstituted or substituted once, twice or three times by halogen, —(C₃-C₆)-cycloalkyl, —(C₂-C₄)-alkenylene, —(C₂-C₆)-alkynyl,
—C(O)—O—R13,
—C(S)—O—R13,
—C(O)—NH—R14 or
—C(S)—NH—R14;
R13 and R14 are each independently
—(C₁-C₆)-alkyl that is unsubstituted or substituted once or twice by —(C₂-C₄)-alkenylene, or —(C₂-C₆)-alkynyl;
R15 is hydrogen,
—(C₁-C₆)-alkyl, or
—SO₂—(C₁-C₆)-alkyl that is unsubstituted or substituted once or twice by —(C₂-C₄)-alkenylene, or —(C₂-C₆)-alkynyl;
R16 is hydrogen,
—(C₁-C₆)-alkyl that is unsubstituted or substituted once or twice by —(C₂-C₄)-alkenylene, or —(C₂-C₆)-alkynyl,
—C(O)—O—R8, or
—O—R8;
p is zero, 1 or 2; and
m is 2; and
n is zero;
or a stereoisomeric form thereof, a mixture of the stereoisomeric forms in any ratio, or a physiologically tolerated salt thereof.

2. The compound according to claim 1, wherein
R1 and R2 together with the carbon atoms to which they are bonded form
a phenyl ring, which is unsubstituted or substituted once or twice by G,
G is hydrogen,
fluorine, chlorine, bromine or iodine,
—(C₁-C₆)-alkyl that is unsubstituted or substituted once, twice or three times by halogen or —(C₂-C₆)-alkynyl,
—C(O)—O—R10,
—C(S)—O—R10,
—C(O)—NH—R11,
—C(S)—NH—R11,
—O—R12,
—C(O)—R10,
—S(O)ₚ—R12,
—NO₂,
—CN,
—N(R15)-R12, or
—SO₂—N(R12)-R16;
R10 is —(C₁-C₆)-alkyl that is unsubstituted or substituted once or twice by —(C₂-C₆)-alkynyl;
R11 is —(C₁-C₆)-alkyl;
R12 is hydrogen,
—(C₁-C₆)-alkyl that is unsubstituted or substituted once, twice or three times by halogen, or —(C₂-C₆)-alkynyl,
—C(O)—O—R13,
—C(S)—O—R13,
—C(O)—NH—R14 or
—C(S)—NH—R14;
R13 and R14 are each independently
—(C₁-C₆)-alkyl that is unsubstituted or substituted once or twice by —(C₂-C₆)-alkynyl;
R15 is hydrogen,
—(C₁-C₆)-alkyl, or
—SO₂—(C₁-C₆)-alkyl that is unsubstituted or substituted once or twice by —(C₂-C₆)-alkynyl;
R16 is hydrogen,
—(C₁-C₆)-alkyl that is unsubstituted or substituted once or twice by —(C₂-C₆)-alkynyl,
—C(O)—O—R8, or
—O—R8;
or a stereoisomeric form thereof, a mixture of the stereoisomeric forms in any ratio, or a physiologically tolerated salt thereof.

3. The compound according to claim 1, which is the compound
2-[4-(4-trifluoromethoxyphenoxy)benzenesulfonyl]-1,2,3,4-tetrahydroisoquinoline-1-hydroxycarboxamide
or a stereoisomeric form thereof, a mixture of the stereoisomeric forms in any ratio, or a physiologically tolerated salt thereof.

4. A pharmaceutical composition comprising a pharmaceutically effective amount of the compound according to claim 1 or a stereoisomeric form thereof, a mixture of the stereoisomeric forms in any ratio, or a physiologically tolerated salt thereof, and a pharmaceutically suitable and physiologically tolerated carrier, additive or excipient.
